(19) United States
(12) Patent Application Publication
(10) Pub. No.: US 2019/0029858 A1
(43) Pub. Date: Jan. 31, 2019

(54) DUODENAL INTERNAL COVERING MEMBRANE FOR DUODENUM

(71) Applicant: Ping WAN, Shanghai (CN)

(72) Inventor: Ping WAN, Shanghai (CN)

(21) Appl. No.: 16/072,122

(22) PCT Filed: Dec. 9, 2016
(86) PCT No.: PCT/CN2016/109284

§ 371 Date: Jul. 23, 2018

(30) Foreign Application Priority Data
Jan. 23, 2016 (CN) 201610043266.3

(51) Int. Cl. A61F 5/00; A61F 2/04

(52) U.S. Cl. CPC A61F 5/0076; A61F 2/04; A61F 2250/0039; A61F 2220/0008; A61F 2002/045

(57) ABSTRACT

Provided is a covering membrane provided within a duodenum. This invention relates to a medical device placed in the digestive tract. At least a part of the duodenal internal covering membrane is prepared from a hydrophobic and oleophobic material and is biodegradable. The duodenal internal covering membrane further includes a biomimetic microarray adhesive sheet, an elastic piece, an anchor hook, and an anti-twist rib. The duodenal internal covering membrane is prepared by electrospinning and can be used to prevent and treat obesity and diabetes.

### BACKGROUND OF THE INVENTION

### Field of the Invention

This invention relates to a gastrointestinal built-in medical instrument and, more particularly, to an internal covering membrane for a duodenum.

### Description of the Related Art

Recently, it has been found that after an obesity patient underwent a gastric bypass surgery to lose weight, not only the body weight is significantly decreased, but also the type 2 diabetes complicated by obesity is alleviated. However, the gastric bypass surgery has clinical risks, such as anastomotic leakage, intestinal obstruction, pulmonary embolism, deep vein thrombosis, portal vein injury and other diseases and death. A duodenal cannula can avoid drawbacks of the gastric bypass surgery and retain its good effects.

In the prior art, a selection of a hydrophilic and/or oleophilic material enables the duodenal cannula or the duodenal internal covering membrane to adhere to an inner wall of an intestine, so as to prevent it from being separated from the intestinal wall and twisting and winding, thereby preventing the “intestinal obstruction” and protecting the function of the cannula or the duodenal internal covering membrane. However, this ignores the blocking of bile and pancreatic juice by the hydrophilic and/or oleophilic intestinal wall adhesion. Continuous bile blockage and poor flow will lead to cholestasis and secondary bile duct infection, congestion and edema of a bile duct mucosa will increase obstruction and increase bile duct pressure, and accumulated bile will gradually become pus; the high-pressure bile duct with purulent bile can cause hepatomegaly, inflammatory changes of intrahepatic bile ducts and surrounding hepatic parenchymal cells, necrosis in large areas of hepatocytes and multiple abscesses in the liver; intrahepatic high-pressure can also cause rupture of intrahepatic capillary bile duct, and the purulent bile or even biliary thrombosis entering blood circulation through intrahepatic sinus, which causes bacteremia and sepsis and even purulent embolism in a lung; and bile duct infection can cause ulcer and biliary tract bleeding, and a series of pathophysiological changes such as septic shock, liver and kidney failure or disseminated intravascular coagulation can occur in late period, and once these changes occur, even if the bile duct blockage and the high-pressure are relieved, the liver parenchyma and the bile duct will still leave irreversible damage. Continuous poor pancreatic juice flow will lead to increased pancreatic pressure, pancreas acini rupture, and trypsinogen entering into interstitium (pancreatic enzymes are often present in the pancreatic cells in the form of inactive trypsin granules), cause the pancreas to digest itself, pancreatic cells and interstitium edema, fat necrosis and hemorrhage; pancreatic cells are damaged, lysosomal hydrolase is released, the trypsinogen is activated, and phospholipase A, elastase and pancreatic kallikrein are further activated to dissolve elastic fibers of vascular wall, which causes damage, rupture, bleeding and necrosis of the pancreatic vessels; and digestive enzymes and necrotic tissue fluid can also be transported throughout the whole body through the blood circulation and a lymphatic pathway, causing damage to the organs in the whole body.

Adhesion to the intestinal wall of the hydrophilic and/or oleophilic material does not prevent the “intestinal obstruction” and does not protect the function of the cannula or duodenal internal covering membrane: there are a variety of digestive juices in the duodenum, and content of eating is various. If there is no double-phobics performance of hydrophobicity and oleophobicity, with continuation of indwelling time in the body (usually at least one month), on the wall of the duodenal cannula, there will be various substances sticking to form scale, which not only reduces the performance of the duodenal cannula, but also becomes a hidden danger of other diseases, and is increasingly block aecretion of digestive juices such as the bile and the pancreatic juice.

The materials of the prior art are undegradable, when removing, an operation process of a second “surgery” is complicated and damages organs, and after removal, it is difficult to avoid a “bounce” problem after the original barrier is instantaneously and completely removed; compared with biomimetic adhesive patch, anchor hook for fixation penetrates into the duodenal bulb, and with peristalsis of the gastrointestinal, loosening, exudation and adhesion are repeated, and when removed, a spike is embedded in intestine tissue and stimulate growth and adhesion of local intestinal tissue; the airtight and impervious duodenal cannula obstructs digestive and absorbing functions of the duodenum and also limits or affects other functions of the intestinal mucosal cells and tissues; weak thermal stability, insufficient mechanical strength and elasticity and easy breakage affect the performance of duodenal cannula; and obviously, a lumen is easy to shrink, twist and wind, which not only affects the function of the cannula, but also interferes with smoothness of the digestive tract.

### BRIEF SUMMARY OF THE INVENTION

The hydrophobic and oleophobic solution according to this invention can solve the problems which are brought by the hydrophilic and/or oleophilic materials in the prior art, and the problems are reducing the function of the duodenal cannula or the duodenal internal covering membrane, blocking the secretion of digestive juice such as bile and pancreatic juice, and inducing other diseases; the complicated operation process of the second “surgery”, the problems of damaging organs and tissues, and the problem of “bounce” during the removal in the prior art can be solved by using a degradable biocompatible material; the solution of the biomimetic adhesive sheet according to this invention can solve the problem that the anchor hook for fixation is embedded into the intestinal tissue which stimulates the growth and adhesion of the local intestinal tissue in the prior art; the electrospinning described in this invention can solve the problem of being non-breathable and moisture impermeable and limiting or affecting other functions of intestinal mucosal cells and tissues in the prior art; a polyurethane material and the like described in this invention can solve the problem of weak thermal stability, insufficient mechanical strength and elasticity, and easy breakage in the prior art; and one or more than one straight-through or spiral and/or irregularly curved anti-twist rib and/or biomimetic adhesive patch according to this invention can solve the problem of shrinkage, twisting, and winding in the prior art.

In the hydrophobic and oleophobic solution, a compound having superhydrophobic and superoleophobic properties such as a short fluorine chain is degradable, and a degradation product is non-toxic and harmless. Steps of prepolymerization, chain-extension and sealing end are optimized to synthesize a perfluoro-terminated fluorine-containing polyurethane, and the structure of the fluorinated polyurethane is determined. The fluorine-terminated polyurethane is introduced into an electrospinning raw liquid. In order to reduce a fiber diameter, the fluorine-terminated polyurethane may also be introduced into a mixture (low boiling point and high boiling point solvents; good solvents and non-good solvents) as a polymer to reduce the concentration of the electrospinning solution. In order to avoid surface adhesion of the fluorinated polyurethane, it can also be modified with nano-SiO₂ particles. Process parameters and environmental parameters of the electrospinning are set to achieve good porosity, strength, wear resistance and so on. A static contact angle of water and oil is >150°, which has excellent water and oil repellency, breathability and moisture permeability. In order to improve the thermal stability, a small amount of perfluoropolyether diol can be added, and/or a content of the perfluoropolyether diol and composition of the hard segment can be adjusted to control Tm of the material.

The diameter and length of a tubular portion are matched with the duodenum and a jejunum in different populations, and the diameter is 10-60 mm. The length is matched with the duodenum and can extend to a segment of the jejunum which is connected with the duodenum, and the length is 80-700 mm. The thickness of the duodenal internal covering membrane of the tubular portion is 0.005-1 mm. The elastic shaped tubular portion is an elastic and/or shape memory material that can prevent the tubular portion from collapsing induced by gastrointestinal peristalsis and reflux of jejunum content caused by the increase of the gap between the duodenal internal covering membrane of the tubular portion and the inner wall of the duodenum. The ampulla portion is a trumpet-shaped portion which is connected with the tubular portion, and the ampulla portion may also be in the shape of a column, a ball, a drum or the like. The ampulla portion has a thickness of 0.005-1 mm and a height of 6-100 mm. The trumpet-shaped portion which is connected with the tubular portion is a progressively open acute angle with an angle of 5 to 65°. Its thickness, height and angle are matched with different populations.

The biomimetic microarray adhesive sheet is obtained from a biocompatible biodegradable or non-biodegradable material and/or a hydrophobic and/or oleophobic material, and may be selected from silicone rubber, polyurethane, multi-wall carbon nanotubes, Polyester resin, polyimide, elastomer, epoxy resin, polydimethylsiloxane, polystyrene, polytetrafluoroethylene, Teflon, polydimethylsiloxane, parylene, Polyurethane and ethylene terephthalate, polymethyl methacrylate and so on or a combination and other known suitable materials. The shape may be a circle, an olive, a trapezoid, a square, a triangle, a cylinder, a diamond or the like, or a combination thereof, and the size may be 1 square nanometer or more, or a combination thereof. The top end of adherent fiber fluff may be curved (shovel-like) or flat-headed or round-headed or layered structure or other shapes and structures or combinations thereof. As an optimization, the biomimetic microarray adhesive sheet may be stitched, adhered, anchored, woven, hooked, riveted, thermoplastic, frozen, pneumatic, electrostatic, etc. and/or a combination thereof or other well known methods and so on, and combinations thereof attached to the duodenal internal covering membrane of the ampulla portion. The precise arrangement may be circular, olive, trapezoidal, square, triangular, cylindrical, diamond-shaped, etc. or a combination thereof, which may be one row or more rows, and the biomimetic microarray adhesive sheet may be close to or be separated or a combination thereof. The binder may be biocompatible polyurethane, polyurethane, silicone, fluorinated ethylene propylene, etc. or a combination thereof or other known materials and a combination thereof.

The biomimetic microarray adhesive sheet can be prepared by using an inductively coupled plasma (ICP) deep etching technique in a microelectromechanical system (MEMS) to form an upright microarray template on a silicon wafer, and it is not excluded to prepare adherent microarrays by other substances and other methods. As an optimization, a synthetic mussel adhesive protein polymer-dopamine-methacrylamide/methoxyethyl acrylate copolymer (P(DMA-co-MEA)) can be synthesized, and other known methods can also be used. The synthesized dopa-containing copolymer is dissolved in methylene chloride solution, the polyurethane microarray is immersed in this solution, and a layer of dopamine-containing copolymer is modified outside the polyurethane microarray. It is not excluded that other substances (including modifiers and modified substances) which have strong adhesion under dry conditions and strong adhesion in water and a preparation method can be formed. As an optimization, the biomimetic microarray adhesive sheet has a suitable contact surface and controls diameter length ratio of the fluff and the spacing of the fluff to avoid mutual adhesion; and as an optimization, the diameter length ratio of the fluff is 0.1-5:20, the length is 0.1-200 μm, and the spacing of the fluff is 0.1-30.0 μm. The preparation process of the biomimetic microarray adhesive sheet can also be performed by atomic force microscopy etching, alumina template hole injection molding, electrostatic induction etching, inductively coupled plasma etching, photolithography (electron beam projection lithography, Nanoimprint lithography, etc.) and can also be prepared by array carbon nanotubes, reactive plasma dry etching, soft etching, directional self-assembly method based on micro-nano fluff growth. The duodenal internal covering membrane and its biomimetic microarray adhesive sheet are soft, smooth, elastic, and have good histocompatibility, no acute systemic reaction, no chronic systemic reaction, no acute local reaction and no chronic local reaction.

The tubular portion and/or the ampulla portion includes a biocompatible biomimetic microarray adhesive sheet for fixation or for maintaining the shape, and/or lines with an anchor hook or without the anchor hook and an elastic piece.

An upper part of the duodenal internal covering membrane may be a wave-shaped or a V-shaped or trapezoidal or wall-shaped ampulla portion elastic film, and the ampulla portion elastic film may include a wave-shaped or a V-shaped or trapezoidal or wall-shaped elastic piece continuously surrounding, and the elastic piece may be prepared by a memory or non-memory biocompatible material. Peaks and valleys of the elastic piece can be single-loop springs and are attached with an anchor hook at intervals, which can fix the ampulla portion, can flex or elastically move according to the movement of the duodenum and the bulb, and can also maintain the shape of the ampulla portion.

The tubular portion and/or the ampulla portion may be respectively thickened and/or reinforced by one or more straight and/or spiral and/or oblique and/or crossed and/or irregularly curved distributed anti-twist rib or a combination of thereof along the longitudinal diameter of the duodenal internal covering membrane in a stitched, adhered, anchored, woven, hooked, riveted, thermoplastic, frozen, pneumatic, electrostatic, etc. way and/or a combination thereof or other known ways. The rib can strengthen, support, expand the duodenal internal covering membrane and prevent twisting and winding of the duodenal internal covering membrane, and have other synergistic and mutual supporting functions with the duodenal internal covering membrane.

The preparation of the ampulla portion and the tubular portion may be electrospinning, electrostatic spraying, casting, laminating, micro-nano process and/or anti-sticking process.

The ampulla portion and the tubular portion may be closed up together or folded into a spherical or cylindrical or a capsule shape or a spindle shape in vitro, the folding manner may be that the distal end of the duodenal internal covering membrane is folded or curled or covered toward the proximal end, and then the ampulla portion is centripetally inverted.

The length, thickness, elasticity, shape, diameter length ratio of the fluff, length of the fluff, diameter of the fluff, spacing of the fluff, etc. of each part of the duodenal internal covering membrane are all reference values, and the actual manufacturing can be specifically designed according to needs.

At least a part of the duodenal internal covering membrane of this invention is hydrophobic and oleophobic, can self-clean in the body, prevents the formation of the scale by sticking various substances on itself, which not only maintains the performance of the duodenal internal covering membrane, but also eliminates the hidden dangers caused by other diseases, and does not block the secretion of digestive juices such as the bile and the pancreatic juice; all of the parts of the duodenal internal covering membrane are prepared by biocompatible materials, that is, to solve the problem of biocompatibility of the materials placed in the body, and to reduce a host reaction generated in the body; and if it is a biodegradable material, it can be gradually degraded in the body after being placed in the body, which can be prepared into a medical equipment for treating diabetes and obesity by reducing damage, preventing falling, avoiding removing and inhibiting bounce.

The biomimetic microarray adhesive sheet of this invention has the advantages of high adhesion, good stability, strong adaptability to materials and shapes, good self-cleaning property, no damage and pollution to intestinal tissues, and functionally support other parts.

The elastic piece of this invention can fix the ampulla portion, flex or elastically move according to the movement of the duodenum and the bulb, and can also maintain the shape of the ampulla portion.

The anti-twist rib and/or the biomimetic adhesive patch of the invention can strengthen, support the duodenal internal covering membrane and prevent twisting and winding, and have other synergistic and mutual supporting functions with the duodenal internal covering membrane.

The electrospinning technology of this invention has good breathability, which can not only anatomically block the contact between food and intestinal mucosa, but also physiologically affect the function of intestinal mucosal cells and tissues as little as possible.

In addition, the duodenal internal covering membrane according to this invention can be made of a material such as polyurethane, which has excellent properties such as high strength, good flexibility, tensile strength, fatigue resistance, wear resistance, certain thermal stability and so on.

### DETAILED DESCRIPTION OF THE INVENTION

This invention will be further described below in combination with specific embodiments:

### Embodiment One

Steps of prepolymerization, chain-extension and sealing end are optimized to synthesize a fluorine end polyurethane, and the structure of the fluorinated polyurethane is determined. The fluorine end polyurethane is dried, then dissolved in dimethylacetamide (DMA), rinsed by methanol-water mixture for several times, purified and dried again.

Polyacrylonitrile (PAN) and fluorinated polyurethane (FPU) are weighed, dimethylformamide (DMF) or DMA/butanone are added, and the FPU is 0.1-1.2 wt % of total mass. The mixture is stirred vigorously at room temperature, the PAN/FPU is 9-16 wt % of the total mass, and the mixture is injected to a syringe pump A. Polyurethane (PU) and FPU are weighed, DMF or DMF/butanone are added, and the FPU is 0.1-1.2 wt % of the total mass. The mixture is vigorously stirred at room temperature, PU/FPU is 9-16 wt % of the total mass, and the mixture is injected to a syringe pump B. In order to avoid surface adhesion of the FPU, it can also be modified with nano-SiO₂ particles, or the PU can be modified by silicone (such as the silicone is introduced into side chain or main chain of Polyurethane, pure soft segment or mixed soft segment and so on). Process parameters and environmental parameters of the electrospinning are set to achieve good porosity, strength, wear resistance, embrittlement resistance, heat resistance, low temperature resistance and so on after testing of tensile property, breathability, wear resistance, contact angle measurement and so on. A static contact angle of water and oil is >150°, which has excellent water and oil repellency, breathability and moisture permeability. In order to improve the thermal stability, a small amount of perfluoropolyether diol can be added, and/or a content of the perfluoropolyether diol and composition of the hard segment can be adjusted to control Tm of the material.

The preparation process can also be prepared by weaving, spinning, laminating, stitching, adhering, anchoring, thermoplastic, blow molding, etching, micro-etching, injection molding, freezing, pneumatic, electrostaticity, stretching, deposition, sol-gel method, template synthesis method, nanoparticle method, phase separation method, self-assembly method, etc. or/and a combination thereof.

### Embodiment Two

The hydrophobic and oleophobic duodenal internal covering membrane according to this invention is cut longitudinally and then cut into a membrane of about 2 cm×2 cm size, and three pieces are taken to be placed in a 100 ml glassware. 60 ml of porridge is taken into the glassware at 8 o'clock every day and is discarded after 1 hour. At 12 o'clock and 18 o'clock on the same day, 30 ml of each of two non-vegetarian laboratory staffs random dishes is taken and placed in the glassware. After 1 hour, 60 ml of the dish is discarded and continue this for 7 days. Another 3 pieces of hydrophilic oleophilic membrane with 2 cm×2 cm size are used for the same operation. At the end of the 7th day, it can be seen that the hydrophilic oleophilic membrane has obvious fouling and even has odor.

### Embodiment Three

As an optimization, the biomimetic microarray adhesive sheet can be prepared by using an inductively coupled plasma (ICP) deep etching technique in a microelectromechanical system (MEMS) to form an upright microarray template on a silicon wafer, the polydimethoxysiloxane (PDMS) is cast onto a silicon template column array, and after solidification, it is stripped and mold released to obtain a polydimethoxysiloxane (PDMS) microarray hole template. Liquid polyurethane or/and other biocompatible materials are cast onto the polydimethoxysiloxane (PDMS) microporous template and solidified and mold released to obtain a polyurethane biomimetic adhesive microarray. It is not excluded to prepare the adherent microarray by other substances and other methods. A synthetic mussel adhesive protein polymer-dopamine-methacrylamide/methoxyethyl acrylate copolymer (P(DMA-co-MEA)) is synthesized; and synthesized dopa-containing copolymer is dissolved in methylene chloride solution, the polyurethane microarray is immersed in this solution, and a layer of the dopamine-containing copolymer is modified outside the polyurethane microarray. It is not excluded that other substances (including modifiers and modified substances) which have strong adhesion under dry conditions and strong adhesion in water and a preparation method can be formed. As an optimization, the biomimetic microarray adhesive sheet has a suitable contact surface and controls diameter length ratio of the fluff and the spacing of the fluff to avoid mutual adhesion. The biomimetic microarray adhesive sheet can be adhered by a biocompatible polyurethane, polyurethane, silicone, fluorinated ethylene propylene and so on, or a combination thereof, or other known materials and a combination thereof. As an optimization, the duodenal internal covering membrane and its biomimetic microarray adhesive sheet are soft, smooth, elastic, and have good histocompatibility, no acute systemic reaction, no chronic systemic reaction, no acute local reaction and no chronic local reaction. As an optimization, the tubular portion of the duodenal internal covering membrane can be adhered or/and woven by adding six spiral anti-twist ribs to strengthen, support, expand the duodenal internal covering membrane and prevent twisting and winding of the duodenal internal covering membrane, and have other synergistic functions with the duodenal internal covering membrane.

### Embodiment Four

The main process steps for preparing the biomimetic microarray adhesive sheet can be: the first step is to use a glow discharge of CF₄ gas to generate activated free radical of F atom. Then the activated free radical of F atom can react with silicon or silica to form silicon tetrafluoride gas, thereby exhibiting an etching effect. The second step: fluorine atoms are introduced into argon plasma, and by using the synergistic effect of the plasma, fluorine and silicon can react quickly, such that the etching effect can be better. The third step: a mask plate pattern is introduced on the silicon wafer, then a column array with a high length diameter ratio is etched on the silicon wafer by using an Oxford ICP180 etching system, and finally the polydimethylsiloxane is cast onto the silicon template column array. This is baken in an oven at 60° C. for 4 h for solidification and is stripped and mold released to obtain a hole array template of the polydimethylsiloxane, and the release mold is peeled off to obtain a pore array template of polydimethylsiloxane. Then other polymer liquids are cast on the polydimethylsiloxane hole array template, and after solidification and mold releasing, a large area micron-sized polymer foot sole bristle adhesive array can be obtained.

### Embodiment Five

Medical polylactic acid, the solvent is a mixed solvent of chloroform/ethanol, the volume ratio is 40/60 (v/v), and the concentration is 4 wt %; and medical polyurethane, the solvent is dimethyl sulfoxide, and the concentration is 10 wt %. The voltage is 17 KV, the propulsion speed is 0.4 ml/h, the receiving distance is 16 cm, and the inner diameter of the pinhole is 0.4 mm. At room temperature 23° C., in a fume hood, the tubular portion is electrospun with the medical polyurethane solution, and the ampulla portion is electrospun with the medical polylactic acid solution.

### Embodiment Six

Twenty healthy male SD rats, weighing 227±14.93 g, are randomly divided into two groups, namely a built-in group and a no built-in group of the duodenal internal covering membrane according to this invention. 0.5 g PEG400 is diluted with 5 ml of normal saline for spare. 50 mg/kg ip Pentobarbital, in the built-in group of the duodenal internal covering membrane, the duodenal internal covering membrane is placed in the duodenum by a pusher. The abdomens of this group are cut open, the beginning end of the jejunum is ligated, the chest of the mouse is raised, and the PEG400 dilution is slowly injected into the lower end of the pylorus by a puncture needle, and the filling is light. Pay attention to intraoperative care. After 30 min, blood is taken from the carotid artery, and PEG400 is measured by high performance liquid chromatography. The data obtained by statistical analysis is analyzed by SPSS 12.0 statistical software package. Analysis of variance is used between groups, t test is used in the group, and the difference is significant at p<0.05. It indicates that the content of the built-in group of the duodenal internal covering membrane is 0.52±0.11 μg/ml, the content of the no built-in group is 5.09±0.87 μg/ml, and p<0.01.

### Embodiment Seven

Twenty healthy male SD rats, weighing 231±16.17 g, are randomly divided into two groups, namely a built-in group and a no built-in group of the duodenal internal covering membrane according to this invention. After 14 days of built-in, two groups of rats are sacrificed, the duodenums are taken, and 3.7% paraformaldehyde is placed, and fixation; the fixed tissues are rinsed by flowing water thoroughly; ascending gradient alcohol is used for dehydration, xylene is used to make the tissues transparent, paraffin wax is used for immersion, paraffin wax embedding is smooth, and excessive wax at the two sides is removed; the tissues are sliced and placed in warm water to spread, complete and wrinkle-free sections/slices are selected and pasted on a slide glass; it is placed in the 55° C. incubator to dry excessive water and paraffin and is dewaxed, and descending gradient alcohol; conventional HE is used staining, ascending gradient alcohol is used for dehydration, xylene is used to make the tissues transparent, and neutral gum is used for sealing; and five discontinuous sections/slices are taken from each rat, each slice is taken in an odd-numbered field of view in sequence, and the small intestine mucosa and its 20 villus are observed. Normal optical microscopy shows that the villus of the two groups of slices are basically normal, and a small amount of partial villus is intermittently widened under the top end. No obvious epithelial peeling, shedding or ulceration of the villus at the top end are detected; and no rupture of the villus at the top end and the epithelial shedding are detected, collapse of lamina propria is not detected, and ulcers and bleeding spots are not detected. The data obtained by statistical analysis is analyzed by SPSS 12.0 statistical software package. Analysis of variance is used between groups, t test is used in the group, and the difference is significant at p<0.05. There is no significant difference between the two groups under the optical microscope(p>0.05).

### Embodiment Eight

Twelve healthy male SD rats, weighing 234±14.65 g, are randomly divided into two groups, namely a built-in group and a no built-in group of the duodenal internal covering membrane according to this invention. After 21 days of built-in, two groups of rats are sacrificed, the duodenums are taken, and 3.7% paraformaldehyde is placed, and fixing; the fixed tissues are rinsed by flowing water thoroughly; ascending gradient alcohol is used for dehydration, xylene is used to make the tissues transparent, paraffin wax is used for immersion, paraffin wax embedding is smooth, and excessive wax at the two sides is removed; the tissues are sliced and placed in warm water to spread, complete and wrinkle-free sections/slices are selected and pasted on a slide glass; it is placed in the 55° C. incubator to dry excessive water and paraffin and is dewaxed, and descending gradient alcohol; conventional HE is used for staining, ascending gradient alcohol is used for dehydration, xylene is used to make the tissues transparent, and neutral gum is used for sealing; and five discontinuous sections/slices are taken from each rat, each slice is taken in an odd-numbered field of view in sequence, and the small intestine mucosa and its 20 villus are observed. Transmission electron microscopy, 8000 times, and 10 random shots per case. Under the transmission electron microscope, no obvious reduction of chorion, lodging of the villus and reduction of villus absorption area are observed. The mitochondria in the intestinal mucosa are basically intact. No mitochondria are observed to be swollen, no nuclear chromatin condensation or nuclear fragmentation is detected, and no obvious apoptotic phenomenon is detected. The image processing software is the specific software DigitalMicrograph of GATAN company. The data obtained by statistical analysis is analyzed by SPSS 12.0 statistical software package. Analysis of variance is used between groups, t test is used in the group, and the difference is significant at p<0.05. There is no significant difference between the two groups (p>0.05).

### Embodiment Nine

Two pieces of latex pads with the thickness of 50 mm correspond to the duodenum and the bending position of the upper part of the jejunum of an adult, respectively, and each of the coronal section is symmetrically hollowed out. After the two latex pads fit together face to face, the middle empty channel therebetween is the channel of the duodenum and upper part of the jejunum of the adult, the proximal end and the distal end of the channel need to be exposed.

The prepared product has a diameter of 10-60 mm, a length of 80-700 mm, a diameter to length ratio of 1:10-30 and a thickness of 0.005-1 mm, and the distal end is marked with a small point by a marker.

The two pieces of latex pads are separated, and the prepared product is put into the duodenum and the bending position of the upper part of the jejunum in one latex pad. The prepared product is rotated 180° clockwise, and the proximal end and the distal end of this product placed into the latex pad are fixed by hand to avoid rotation and restoring.

The other latex pad is aligned with the channel of the previous latex pad, and the two are closed.

The hand that fixes the distal end of this product is immediately released, and it shows that the rotation is restored after 1-2 seconds.

The channels of the latex pad with different sizes are respectively rotated 90° or 360° counterclockwise or clockwise, respectively, and the latex pads are erected or horizontally placed or irregularly danced, respectively, which all shows that the rotation is rapidly restored.

### Embodiment Ten

The duodenum to the jejunum of a fresh pig is taken, and the prepared product (the distal end is marked with a small point by the marker) is placed into the duodenum to the jejunum of the pig. The product is rotated 180° clockwise and loosely placed into one latex pad that has been hollowed out (corresponding to the duodenum and the bending position of the upper part of the jejunum of the adult). The proximal end and the distal end of this product placed into the duodenum to the jejunum of the pig are fixed by hand to avoid rotation and restoring.

The hand that fixes the distal end of this product is immediately released, and it shows that the rotation is restored after 1-2 seconds.

It is rotated 90° or 360° counterclockwise or clockwise, respectively, and it shows that the rotation is rapidly restored.

The parts to which the invention does not relate contain the same contents with the prior art or can be implemented by adopting the prior art.

What is claimed is:

1. A duodenal internal covering membrane, wherein the duodenal internal covering membrane is prepared by a biocompatible material, and at least a part of the duodenal internal covering membrane is prepared by a hydrophobic and oleophobic material.

2. The duodenal internal covering membrane according to claim 1, wherein the duodenal internal covering membrane comprises an ampulla portion and a tubular portion, the tubular portion communicates with the ampulla portion, and both the ampulla portion and the tubular portion are prepared by the hydrophobic and oleophobic material.

3. The duodenal internal covering membrane according to claim 2, the duodenal internal covering membrane further comprising a biomimetic microarray adhesive sheet attached to the ampulla portion and/or the tubular portion so as to fix the ampulla portion and/or the tubular portion to a duodenum and a bulb thereof.

4. The duodenal internal covering membrane according to claim 2, wherein the duodenal internal covering membrane further comprises an elastic piece fixed at the ampulla portion to maintain a shape of the ampulla portion.

5. The duodenal internal covering membrane according to claim 4, wherein the elastic piece has an anchor hook, and the anchor hook is fixed at the duodenal bulb.

6. The duodenal internal covering membrane according to claim 2, wherein the duodenal internal covering membrane further comprises one or more than one anti-twist ribs disposed at the tubular portion.

7. The duodenal internal covering membrane according to claim 6, wherein a plurality of anti-twist ribs extend along an axis direction parallel to the tubular portion and are parallel to each other.

8. The duodenal internal covering membrane according to claim 6, wherein a plurality of anti-twist ribs are distributed along the tubular portion in a helical and/or oblique and/or cross-shaped and/or irregularly curved way.

9. The duodenal internal covering membrane according to claim 1, wherein the duodenal internal covering membrane is prepared by an electrospinning process.

10. The duodenal internal covering membrane according to claim 1, when used in the prevention or treatment of obesity or diabetes.
